(19) United States
(12) Patent Application Publication
(10) Pub. No.: US 2009/0068059 A1
(43) Pub. Date: Mar. 12, 2009

(54) METHOD OF PREVENTING HYDROGEN SULFIDE ODOR GENERATION IN AN AQUEOUS MEDIUM

(76) Inventors: Wilson K. Whitekettle, Jamison, PA (US); Gloria J. Tafel, Doylestown, PA (US)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 11/854,133

(22) Filed: Sep. 12, 2007

(51) Int. Cl. A61L 9/05; C23F 11/08

(52) U.S. Cl. 422/5; 422/7

(57) ABSTRACT

The present invention relates to a fast acting chemical treatment for preventing the generation of hydrogen sulfide odor by the microbial metabolic activities of sulfate reducing bacteria. Specifically, the invention relates to a method for preventing hydrogen sulfide odor generation in a sulfur species-containing aqueous medium, which includes adding to the aqueous medium an effective amount for the purpose of a sulfide generation inhibiting treatment including chlorhexidine and salts thereof.

### FIELD OF THE INVENTION

The present invention relates to a fast-acting, aqueous phase chemical treatment for preventing the generation of hydrogen sulfide odor in an aqueous medium.

### BACKGROUND OF THE INVENTION

The reactivity between various aldehydes and sulfidic compounds (H₂S, mercaptans, etc.) has been known in the art for some time. For example, Marks in U.S. Pat. No. 1,991,765 discloses a method of reacting hydrogen sulfide and an aldehyde in an aqueous solution having a pH between 2 and 12, and at a temperature between substantially 20° C. and 100° C. After Marks' disclosure in 1935, many patents appeared disclosing the use of aldehydes during acid cleaning of iron sulfide deposits, including U.S. Pat. Nos. 2,606,873; 3,514,410; 3,585,069; 3,669,613; 4,220,550; 4,289,639; and 4,310,435. Consumption of the hydrogen sulfide liberated by acidification of sulfide-containing deposits increased the safety of such operations. Decreased corrosivity of the aldehyde-containing acids is also disclosed in the prior art, sometimes with the addition of ancillary corrosion inhibitors.

Menaul in U.S. Pat. No. 2,426,318 discloses a method of inhibiting the corrosive action of natural gas and oil containing soluble sulfides on metals by utilizing an aldehyde, preferably formaldehyde.

Roehm in U.S. Pat. No. 3,459,852 discloses a process for deodorizing and reducing the biochemical demand of an aqueous solution which contains at least one compound of hydrogen sulfide and compounds containing the —SH group. Roehm's process comprises mixing the solution with a sulfide-active alpha, beta unsaturated aldehyde or ketone in an amount sufficient to form a sulfur-containing reaction product of the sulfide active aldehyde or ketone. Two such sulfide-active compounds disclosed by Roehm are acrolein and 3-buten-2-one.

Formaldehyde, formaldehyde with SO₃⁻², and acrolein are all commercially used hydrogen sulfide (H₂S) scavengers. However, formaldehyde produces a solid reaction product and reverts readily to formaldehyde and free H₂S. Acrolein is more expensive than formaldehyde as well as extremely toxic and dangerous to handle. The use of SO₃⁻² with formaldehyde eliminates the re-release of H₂S but not solids formation.

The goal of many industries is to reduce or eliminate odors and corrosion resulting from the chemical action of sulfides and hydrogen sulfide gas generated by microbiological activity. The objectionable, and possibly toxic gas is often generated by the anaerobic group of microorganisms known as sulfate reducing bacteria (SRB). In municipal and industrial wastewater treatment, it is additionally desirable to inhibit sulfide production by the SRB, while not inhibiting the normal biodegradation processes being performed by the beneficial microbial populations. Addition of chemical species that act as very specific inhibitors of the sulfide generation process would accomplish the desired goals of inhibiting sulfide production (sulfate reduction process) while not interfering with the biochemical reactions and viability of the general microbial populations. These needs are accomplished by the treatments of the present invention.

### SUMMARY OF THE INVENTION

The present invention relates to a fast acting chemical treatment for preventing the generation of hydrogen sulfide odor by the microbial metabolic activities of sulfate reducing bacteria. Specifically, the invention relates to a method for preventing hydrogen sulfide odor generation in a sulfur species-containing aqueous medium, which comprises adding to the aqueous medium an effective amount for the purpose of a chlorhexidine salt, sulfide generation inhibiting treatment. Chlorhexidine digluconate is a preferred treatment.

The present invention further relates to a method for controlling corrosion in a hydrogen sulfide-containing aqueous medium, which method comprises adding to the aqueous medium an effective amount for the purpose of the treatment noted above.

Many chemical compounds are known toxic agents to microorganisms and are known to inhibit sulfate reducing bacteria, resulting in inhibition of generated H₂S. However, these compounds are not specific in their antimicrobial activity, and also inhibit or kill beneficial microorganisms. In the treatment of wastewaters, this could be detrimental to their operations as well as the environment, as non-specific toxics discharged from municipal or industrial wastewater systems can have negative effects on non-target aquatic organisms.

The treatments of the present invention are specific inhibitors of H₂S and sulfide generation, and do not exhibit toxicity to other microbial forms when used in concentrations inhibitory to SRB. Use of these treatments allows for sulfide generation inhibition, while maintaining the overall health of e.g., a wastewater treatment plant, a pulping and papermaking medium, a cooling water medium or a fluid transporting pipeline.

### DESCRIPTION OF THE PREFERRED EMBODIMENTS

The present invention utilizes chemicals which can act as redox potential buffers to treat a liquid medium containing microorganisms (such as but not limited to sulfate reducing bacteria, or SRB), which can generate H₂S odor by converting sulfur species of higher oxidation states to sulfides (S²⁻). By the addition of a small but effective amount of such agents to the liquid (e.g., wastewater, cooling water or water/hydrocarbon emulsion) medium, the sulfide is not microbiologically produced. These agents are introduced in order to inhibit sulfate reducing bacteria and maintain the system redox potential at a level which is less favorable for the microbials to produce H₂S. They function to: (1) efficiently stop H₂S generation by inhibiting the anaerobic microbiological conversion of sulfate to sulfide by SRB; and (2) provide additional redox potential adjustment/maintenance by introducing mild oxidant and/or alternative nutrient sources for the microorganisms in the system.

Since H₂S is a highly corrosive gas, preventing H₂S from forming in systems such as cooling systems and storage tanks also provides an effective means of corrosion control. Further, in a preferred embodiment, the present invention relates to a method for preventing hydrogen sulfide odor generation in a hydrogen sulfide-containing aqueous medium, which comprises adding to the aqueous medium an effective amount for the purpose of chlorhexidine digluconate. About 10 ppm of the treatment is preferably added to the aqueous medium, with an amount of treatment of from about 2-6 ppm being particularly preferred.

The invention will be further illustrated by the following examples, which are included as being illustrations of the invention and which should not be construed as limiting the scope thereof.

### EXAMPLES

The test protocol was as follows: 100 mL serum bottles were filled with synthetic nutrient medium, then stoppered, capped, and autoclaved, and allowed to cool to at least 35 ° C. Using a 1 mL syringe with needle, the treatment was added at desired dosages to prepared serum bottles (triplicates of each dosage). No treatment was added for baseline controls. At least 6 baseline serum bottles were used. Using a 1 mL syringe with needle, 0.1 mL of prepared culture was added to each serum bottle.

Next, a 10¹⁰ cells/ml concentration of the sulfate reducers was prepared (using autoclaved synthetic nutrient medium) inside an anaerobic chamber. Using a 1 ml syringe with needle, 0.1 ml of the prepared sulfate reducers culture was added to each serum bottle. The bottles were then incubated at the desired temperature.

The baseline dissolved sulfide levels were then measured to determine if the samples were ready for efficacy analysis. Using a 30 mL syringe with needle, 30 mL of sample was removed from each serum bottle. About 25 mL of the sample was added to a 30 mL bottle containing 0.125 mL zinc acetate solution, followed by adding 1 mL of 1N NaOH. The sulfide in the fixed sample was then measured. The remaining 5 mL of sample in the syringe was used for pH determination.

The treatments of the present invention are non-biocidal in nature, i.e., antimicrobial activities are not irreversibly affected after treatment. Furthermore, the treatments of the present invention do not result in a significant alteration of medium pH at appropriate dosages. Note that at application dosages for all chemical treatments listed, approximately 100% inhibition of H₂S production was achieved.

TABLE I
Control of H₂S and Sulfate Reducing Bacteria (SRB) by
Salts of Chlorhexidine - Inhibition of SRB by Chlorhexidine Salts

| | | SRB | |
|---|---|---|---|
| | Inhibitor | concentration | |
| Sample | Concentration | (log. viable | |
| Identification | (ppm) | cells/ml) | % H₂S Inhibition |
| Control | 0 | 1.00E+08 | 0 |
| Control | 0 | 1.00E+07 | 0 |
| Control | 0 | 1.00E+08 | 0 |
| Chlorhexidine | 25 | 1.00E+03 | 99 |
| digluconate | 25 | 1.00E+03 | 99 |
| | 25 | 1.00E+04 | 99 |
| | 50 | 1.00E+03 | 99 |
| | 50 | 1.00E+02 | 99 |
| | 50 | 1.00E+02 | 99 |
| | 100 | 1.00E+01 | 99 |
| | 100 | 0.00E+00 | 100 |
| | 100 | 1.00E+02 | 99 |
| Chlorhexidine | 25 | 1.00E+03 | 99 |
| dihydrochloride | 25 | 1.00E+04 | 99 |
| | 25 | 1.00E+04 | 99 |
| | 50 | 1.00E+03 | 99 |
| | 50 | 1.00E+02 | 99 |
| | 50 | 1.00E+02 | 99 |
| | 100 | 0.00E+00 | 100 |
| | 100 | 0.00E+00 | 100 |
| | 100 | 1.00E+01 | 99 |

As shown above, salts of chlorhexidine greatly inhibit the growth of SRB bacteria at concentrations of greater than 25 ppm. Hydrogen sulfide can be greatly reduced by inhibiting the organisms responsible for the generation. However, salts of chlorhexidine can also be toxic to beneficial non-hydrogen sulfide producing organisms at concentrations greater than 25 ppm. Table II (below) shows that the production of hydrogen sulfide by SRB can be essentially eliminated with the use of salts of chlorhexidine at concentrations of about 1.0-5.0 ppm. At these reduced chlorhexidine concentrations, there is little to no toxicity to target or non-target organisms, while demonstrating inhibition of hydrogen sulfide generation. These results occur within about 3° C. to about 35° C., although the present invention is expected to be effective within a temperature range of from about 0° C. to about 50° C. It is anticipated that additional materials, such as chlorhexidine dihydrate, chlorhexidine disulfate and chlorhexidine diacetate will also be effective.

TABLE II
Control of H₂S and Sulfate Reducing Bacteria (SRB) by
Salts of Chlorhexidine - Inhibition of SRB by Chlorhexidine Salts

| | Inhibitor | H₂S | |
|---|---|---|---|
| | Concentration | concentration | |
| Sample Identification | (ppm) | (ppm) | % Inhibition |
| Control | 0 | 10 | 0 |
| Control | 0 | 9.8 | 0 |
| Control | 0 | 10.2 | 0 |
| Control | 0 | 9.7 | 0 |
| Control | 0 | 10.1 | 0 |
| Chlorhexidine | 1 | 0 | 100 |
| digluconate | 1 | 0 | 100 |
| | 1 | 0 | 100 |
| | 2.5 | 0 | 100 |
| | 2.5 | 0 | 100 |
| | 2.5 | 0 | 100 |
| | 5 | 0 | 100 |
| | 5 | 0 | 100 |
| | 5 | 0 | 100 |
| Chlorhexidine | 1 | 0.4 | 96 |
| dihydrochloride | 1 | 0.2 | 98 |
| | 1 | 0.5 | 95 |
| | 2.5 | 0 | 100 |
| | 2.5 | 0 | 100 |
| | 2.5 | 0 | 100 |
| | 5 | 0 | 100 |
| | 5 | 0 | 100 |
| | 5 | 0 | 100 |

In a further preferred embodiment of the present invention, the pH of the aqueous medium is from about 3.5 to about 9.0, with a pH of the medium of about 6-8 being particularly preferred.

While the present invention has been described with respect to particular embodiment thereof, it is apparent that numerous other forms and modifications of the invention will be obvious to those skilled in the art. The appended claims and this invention generally should be construed to cover all such obvious forms and modifications, which are within the true spirit and scope of the present invention.

We claim:

1. A method for preventing hydrogen sulfide odor generation in a sulfur species-containing aqueous medium, which comprises adding to the aqueous medium an effective amount for the purpose of a chlorhexidine salt, sulfide generation inhibiting treatment.

2. The method as recited in claim 1, wherein said aqueous medium has a pH of about 3.5-9.0.

3. The method as recited in claim 1, wherein the addition is conducted at a temperature of from about 0° C. to about 50° C.

4. The method as recited in claim 3, wherein the addition is conducted at a temperature of from about 3° C. to about 35° C.

5. The method as recited in claim 2, wherein said aqueous medium has a pH of about 6-8.

6. The method as recited in claim 1, wherein said aqueous medium comprises a wastewater medium or a pulping and papermaking medium.

7. The method as recited in claim 1, wherein said aqueous medium comprises a cooling water medium or a fluid transporting pipeline.

8. The method as recited in claim 1, wherein said chlorhexidine salt is chlorhexidine dihydrochloride.

9. The method as recited in claim 1, wherein said chlorhexidine salt is selected from the group consisting of chlorhexidine dihydrate, chlorhexidine disulfate and chlorhexidine diacetate.

10. A method for preventing hydrogen sulfide odor generation in a sulfur species-containing aqueous medium, which comprises adding to the aqueous medium an effective amount for the purpose of a chlorhexidine digluconate, sulfide generation inhibiting treatment.

11. The method as recited in claim 10, wherein said aqueous medium has a pH of about 3.5-9.0.

12. The method as recited in claim 10, wherein the addition is conducted at a temperature of from about 0° C. to about 50° C.

13. The method as recited in claim 12, wherein the addition is conducted at a temperature of from about 3° C. to about 35° C.

14. The method as recited in claim 10, wherein said aqueous medium has a pH of about 6-8.

15. The method as recited in claim 10, wherein said aqueous medium comprises a wastewater medium or a pulping and papermaking medium.

16. The method as recited in claim 10, wherein said aqueous medium comprises a cooling water medium or a fluid transporting pipeline.

17. A method for controlling corrosion in a sulfur species-containing aqueous medium, which comprises adding to the aqueous medium an effective amount for the purpose of a chlorhexidine salt.

18. The method as recited in claim 17, wherein said aqueous medium has a pH of about 3.5-9.0.

19. The method as recited in claim 18, wherein said aqueous medium has a pH of about 6-8.

20. The method as recited in claim 17, wherein the addition is conducted at a temperature of from about 0° C. to about 50° C.
